(19) United States
(12) Patent Application Publication
(10) Pub. No.: US 2026/0347285 A1
(43) Pub. Date: Nov. 5, 2026

(54) ACETATE ABSORBER IN OXIDATIVE DEHYDROGENATION SYSTEM

(71) Applicant: NOVA CHEMICALS (INTERNATIONAL) S.A., Fribourg (CH)

(72) Inventors: Shahin GOODARZNIA, Calgary (CA); Vasily SIMANZHENKOV, Calgary (CA); Bolaji OLAYIWOLA, Calgary (CA)

(73) Assignee: NOVA CHEMICALS (INTERNATIONAL) S.A., Fribourg (CH)

(21) Appl. No.: 19/120,390

(22) PCT Filed: Oct. 2, 2023
(86) PCT No.: PCT/IB2023/059876

§ 371 Date: Apr. 11, 2025

Related U.S. Application Data
(60) Provisional application No. 63/415,484, filed on Oct. 12, 2022.

(51) Int. Cl. C07C 5/48; B01D 53/14; B01D 53/18; B01D 53/72; B01D 53/78; B01D 53/96; B01J 23/28; B01J 23/887; C07C 7/11

(52) U.S. Cl. CPC C07C 5/48; B01D 53/1418; B01D 53/1425; B01D 53/1487; B01D 53/1493; B01D 53/18; B01D 53/72; B01D 53/78; B01D 53/96; B01J 23/28; B01J 23/8877; C07C 7/11; B01D 2251/70; B01D 2256/24; B01D 2257/7022

(57) ABSTRACT

A process and a system for oxidative dehydrogenation (“ODH”) of hydrocarbons are provided. An exemplary process includes contacting an ethane feed with an oxidant in the presence of an oxidative dehydrogenation catalyst in an oxidative dehydrogenation reactor under oxidative dehydrogenation conditions to produce an output stream. The ethane feed includes ethane. The output stream includes ethylene, acetic acid, and residual ethane from the ethane feed. The process includes contacting the output stream with an acetate in an absorber to separate a product stream from a residual ethane stream. The product stream includes the ethylene. The residual stream includes the residual ethane from the ethane feed.

### TECHNICAL FIELD

The present specification is directed to an oxidative dehydrogenation process to convert ethane to ethylene. More specifically, a process and system integrating an oxidative dehydrogenation process with acetate absorption for purification of the product ethylene are described.

### BACKGROUND ART

Olefins like ethylene, propylene, and butylene are basic building blocks for a variety of commercially valuable polymers. Since naturally occurring sources of olefins do not exist in commercial quantities, polymer producers rely on methods for converting the more abundant lower alkanes into olefins. To produce ethylene commercial scale producers typically use steam cracking, an energy intensive process that requires extensive downstream separation and is subjected to periodic shutdowns for cleaning and maintenance related to the buildup of coke by-products within the cracking infrastructure. An alternative method is oxidative dehydrogenation (“ODH”), where the lower alkane, such as ethane, is mixed with oxygen in the presence of a catalyst to produce the corresponding olefin, such as ethylene. The ODH reaction couples endothermic removal of hydrogen from the lower alkane with exothermic oxidation of hydrogen to produce water. Unfortunately, the production of byproducts, such as acetic acid, may accompany the production of the target alkene.

Separation of the produced ethylene from residual ethane for traditional steam cracking typically employs cryogenic distillation. While effective, cryogenic distillation can require large and energy intensive towers. Reducing the operational and capital costs of the separation of ethylene from ethane can be beneficial.

### SUMMARY OF INVENTION

Certain aspects of the subject matter described can be implemented as a process for the oxidative dehydrogenation of ethane. The process includes contacting an ethane feed with an oxidant in the presence of an oxidative dehydrogenation catalyst in an oxidative dehydrogenation reactor under oxidative dehydrogenation conditions to produce an output stream. The ethane feed includes ethane. The output stream includes ethylene, acetic acid, and residual ethane. The process includes contacting the output stream with an acetate in an absorber to separate a product stream from a residual ethane stream. The product stream includes the ethylene. The residual ethane stream includes the residual ethane from the ethane feed.

This, and other aspects, can include one or more of the following features.

In some embodiments, prior to contacting the output stream with the acetate in the absorber, the output stream is cooled to a temperature at which the ethylene forms a complex with the acetate in response to contacting the acetate.

In some embodiments, an ethylene-acetate complex is formed in the absorber in response to the output stream contacting the acetate in the absorber. In some embodiments, the process includes decoupling the ethylene-acetate complex in an acetate regenerator to form the product stream.

In some embodiments, prior to contacting the output stream with the acetate in the absorber, the output stream is directed to an acetic acid scrubber upstream of the absorber. In some embodiments, a side stream is separated from the output stream using the acetic acid scrubber. The side stream can include at least a first portion of the acetic acid. In some embodiments, the side stream is directed to the absorber.

In some embodiments, decoupling the ethylene-acetate complex in the acetate regenerator forms an acetate stream that includes water and the acetate.

In some embodiments, the acetate stream is directed to an extractor to separate an aqueous acetic acid stream and a diluted acetate stream.

In some embodiments, the diluted acetate stream is directed to a dryer to separate a recycle stream. The recycle stream can include the acetate. In some embodiments, the recycle stream is directed to the absorber.

In some embodiments, prior to directing the output stream to the acetic acid scrubber, the output stream is directed to a quench tower upstream of the acetic acid scrubber. In some embodiments, a bottom stream is separated from the output stream using the quench tower. The bottom stream can include at least a second portion of the acetic acid. In some embodiments, the bottom stream is directed to the extractor.

In some embodiments, the oxidative dehydrogenation catalyst includes molybdenum, vanadium, oxygen, and an element chosen from at least one of iron, aluminum, or beryllium.

In some embodiments, the acetate is copper acetate. In some embodiments, the acetate is silver acetate.

In some embodiments, at least a portion of the residual ethane stream is directed through a demethanizer to remove carbon monoxide and methane.

In some embodiments, at least a portion of the residual ethane stream is directed downstream of the demethanizer to a C2 splitter to separate an ethane stream.

Certain aspects of the subject matter described can be implemented as a system for the oxidative dehydrogenation of ethane. The system includes a feed stream including ethane and an oxidant. The system includes an oxidative dehydrogenation reactor configured to receive the feed stream. The oxidative dehydrogenation reactor includes an oxidative dehydrogenation catalyst. The oxidative dehydrogenation reactor is configured to contact the feed stream with the oxidative dehydrogenation catalyst under oxidative dehydrogenation conditions to produce an output stream that includes ethylene, acetic acid, and residual ethane from the feed stream. The system includes an absorber including acetate. The absorber is configured to receive the output stream and separate a product stream from the output stream. The acetate is configured to complex with the ethylene to form an ethylene-acetate complex. The product stream includes the ethylene.

This, and other aspects, can include one or more of the following features.

In some embodiments, the system includes a quench tower downstream of the oxidative dehydrogenation reactor and upstream of the absorber. In some embodiments, the quench tower is configured to receive the output stream and cool the output stream to a temperature at which the ethylene forms the ethylene-acetate complex with the acetate in response to contacting the acetate in the absorber.

In some embodiments, the system includes an acetic acid scrubber upstream of the absorber and downstream of the quench tower. In some embodiments, the acetic acid scrubber is configured to receive the output stream from the quench tower and separate the output stream into a side stream and an acetic acid scrubber exit stream. The side stream can include at least a first portion of the acetic acid from the output stream. The acetic acid scrubber exit stream can include the ethylene and the residual ethane from the feed stream. In some embodiments, the absorber is configured to receive the side stream and the acetic acid scrubber exit stream.

In some embodiments, the absorber includes a regenerator configured to decouple the ethylene-acetate complex to form the product stream and an acetate stream that includes water and the acetate.

In some embodiments, the system includes a demethanizer configured to receive at least a portion of the outlet stream separated from the product stream. In some embodiments, the demethanizer is configured to separate the portion of the outlet stream separated from the product stream into a first demethanizer exit stream and a second demethanizer exit stream. The first demethanizer exit stream can include carbon monoxide and methane. The second demethanizer exit stream can include ethane and ethylene.

In some embodiments, the system includes a C2 splitter configured to receive the second demethanizer exit stream. In some embodiments, the C2 splitter is configured to separate the second demethanizer exit stream into a first C2 splitter exit stream and a second C2 splitter exit stream. The first C2 splitter exit stream can include ethane. The second C2 splitter exit stream can include ethylene.

In some embodiments, the system includes an extractor configured to receive the acetate stream and separate the acetate stream into an aqueous acetic acid stream and a diluted acetate stream.

In some embodiments, the system includes a dryer configured to receive the diluted acetate stream to separate the diluted acetate stream into a recycle stream and a dryer exit stream. The recycle stream can include the acetate. The dryer exit stream can include water. In some embodiments, the absorber is configured to receive the recycle stream from the dryer.

In some embodiments, the system includes a dryer outlet connected to the dryer; the dryer outlet is configured to receive the recycle stream from the dryer; and the dryer outlet is connected to the absorber as a copper acetate feed inlet.

In some embodiments, the quench tower is configured to contact the output stream with a quench stream to cool the output stream. In some embodiments, the quench tower is configured to discharge a bottom stream including at least a portion of the acetic acid from the output stream. In some embodiments, the extractor is configured to receive the bottom stream from the quench tower.

In some embodiments, the oxidative dehydrogenation catalyst includes molybdenum, vanadium, oxygen, and an element chosen from at least one of iron, aluminum, and beryllium.

In some embodiments, the acetate of the absorber is copper acetate.

In some embodiments, the acetate of the absorber is silver acetate.

The details of one or more embodiments of the invention are set forth in the accompanying drawings and the following description. Other features, objects, and advantages of the invention will be apparent from the description and drawings, and from the claims.

### BRIEF DESCRIPTION OF THE FIGURES

FIG. 1 is a block diagram of an example ethane oxidative dehydrogenation (ODH) system.

FIGS. 2A and 2B are block diagrams of an example of an ethane ODH system.

FIG. 3 is a block flow diagram of an example method for implementing an acetate absorber in an ethane ODH system.

FIG. 4 is a process flowsheet for implementing an acetate absorber in an ethane ODH system.

### DESCRIPTION OF EMBODIMENTS

Provided herein are a process and system for the oxidative dehydrogenation (ODH) of ethane into ethylene. The process described includes oxidatively dehydrogenating the ethane into ethylene in an ODH reactor in the presence of oxygen and an ODH catalyst and under ODH conditions to form an output stream comprising ethylene, residual ethane, and acetic acid, and cooling the output stream below an ethylene-acetate complexation temperature before introduction into an acetate absorber to separate the ethylene from residual ethane and other components present in the output stream. The process and system provide an integration opportunity which allows removal of cryogenic separation units downstream of an ODH reactor, reducing costs and providing energy savings, and permits mitigation of absorbent degradation as acetic acid recovered from the output stream can be used to replenish degraded acetate in the acetate absorber.

### The Ethane ODH System

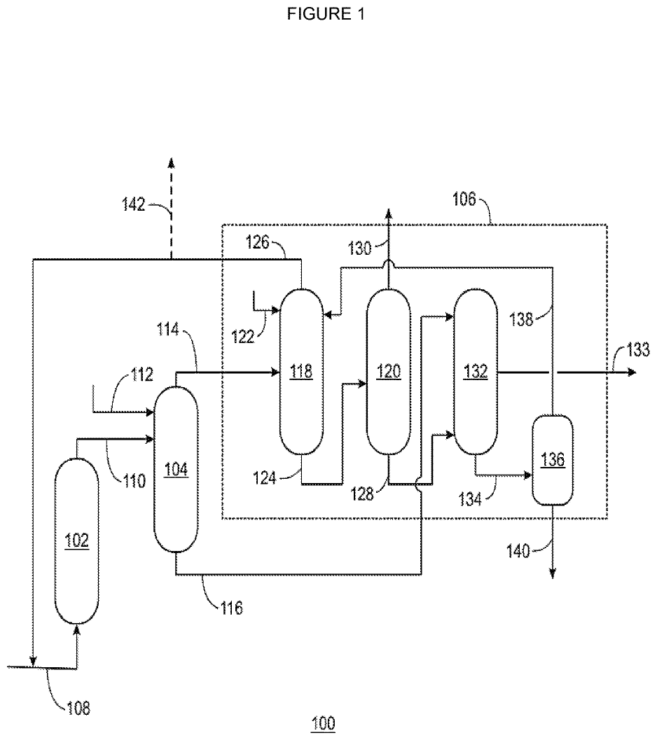

FIG. 1 is a block diagram of an ODH system 100 for the ODH of ethane. The ethane ODH system 100 comprises an ODH reactor 102, a quench tower 104, and an absorber 106. The ODH reactor 102 is configured to receive a feed stream 108. The feed stream 108 includes an oxidant (such as oxygen) and ethane. In some embodiments, the oxidant and the ethane can be added separately to the ODH reactor 102, and the oxidant and the ethane mix within the ODH reactor 102 to form the feed stream 108. The ODH reactor 102 includes an ODH catalyst. The ethane and the oxidant react in the presence of the ODH catalyst under ODH conditions to produce an output stream 110. The output stream 110 includes ethylene and acetic acid. In some cases, the output stream 110 also includes ethane from the feed stream 108 that remains unreacted (residual ethane).

The quench tower 104 is configured to receive the output stream 110 from the ODH reactor 102 and cool the output stream 110. In some embodiments, the quench tower 104 is configured to receive a quench stream 112, which can be brought into contact with the output stream 110 to cool the output stream 110. The quench stream 112 can include, for example, water. The quench stream 112 coming into contact with the output stream 110 in the quench tower 104 causes components in the output stream 110 (such as acetic acid) to condense and drop out of the output stream 110. The quench stream 112 can also scrub a portion of the acetic acid present in the output stream 110. The quench tower 104 can be configured to discharge a bottom stream 116. The bottom stream 116 is the liquid stream exiting the quench tower 104 and includes the acetic acid that has condensed from the output stream 110 and water (for example, from the output stream 110 and the quench stream 112). The cooled outlet stream 114 is the gas stream exiting the quench tower 104 and includes the ethylene and the residual ethane. The cooled outlet stream 114 may include acetic acid, but the cooled outlet stream 114 exiting the quench tower 104 has a decreased acetic acid content in comparison to the outlet stream 110 entering the quench tower 104.

The absorber 106 includes an acetate contactor 118 and an acetate regenerator 120. The acetate contactor 118 includes an acetate that is configured to complex with the ethylene present in the cooled outlet stream 114 via double bonds in response to coming into contact with the ethylene. The acetate can be, for example, copper (Cu) acetate or silver (Ag) acetate. The acetate contactor 118 is configured to receive the outlet stream 114 from the quench tower 104. The outlet stream 114 can be processed prior to entering the acetate contactor 118. The outlet stream 114 can be processed to remove carbon dioxide, for example, by an amine tower including an amine solvent. Then the outlet stream 114 can be compressed and cooled, such that the operating temperature of the cooled outlet stream 114 facilitates the formation of an ethylene-acetate complex in the acetate contactor 118. The residual ethane does not form a complex with the acetate in the acetate contactor 118 and exits the acetate contactor 118 as a residual ethane stream 126. The residual ethane stream 126 can be recycled to the ODH reactor 102 to increase ethylene production. In some embodiments, acetic acid 122 is flowed to the acetate contactor 118. Providing additional acetic acid to the acetate contactor 118 can improve complexing of the ethylene with the acetate to form the ethylene-acetate complex in the acetate contactor 118. For example, the additional acetic acid can aid in replenishing the acetate in the acetate contactor 118 for complexing with the ethylene. As another example, the additional acetic acid can aid in pH control in the acetate contactor 118. As another example, the additional acetic acid can aid in mitigating and/or eliminating the risk of fouling by acetate degradation. The ethylene-acetate complex, acetic acid, and water can exit the acetate contactor 118 via an acetate complex stream 124.

The acetate regenerator 120 is configured to receive the acetate complex stream 124 from the acetate contactor 118. The acetate regenerator 120 is configured to decouple the ethylene-acetate complex of the acetate complex stream 124 to reform the ethylene and the acetate. In effect, the acetate regenerator 120 separates the ethylene as a high purity product stream 130 from a remaining portion of the acetate complex stream 124 (for example, acetate, acetic acid, and water), which exits the acetate regenerator 120 via a regenerator exit stream 128.

In some embodiments, the absorber 106 includes an acetic acid separator 132. The acetic acid separator 132 is configured to receive the regenerator exit stream 128 from the acetate regenerator 120 and the bottoms stream 116 from the quench tower 104. The acetic acid separator 132 includes an extractant that is configured to extract acetic acid from the water of the regenerator exit stream 128. The extractant can be, for example, Cu-acetate-based, sodium (Na) acetate-based, or methyl tert-butyl ether (MTBE)-based. The acetic acid exits the acetic acid separator 132 via an acetic acid stream 133. The acetic acid stream 133 can, for example, be further processed and/or sold. The acetic acid stream 133 can, for example, be used as an intermediate for other products, such as vinyl acetate or ammonium acetate. In some cases, a portion of the acetic acid stream 133 can be recycled to the acetate contactor 118, for example, in addition to or instead of the acetic acid 122 for pH balance, as needed. The acetate and water exits the acetic acid separator 132 via a diluted acetate stream 134.

In some embodiments, the absorber 106 includes an acetate knock off drum 136. The acetate knock off drum 136 is configured to receive the diluted acetate stream 134. The acetate knock off drum 136 is configured to concentrate the acetate by separating the acetate from the water in the diluted acetate stream 134. For example, the acetate knock off drum 136 can include a dryer that boils off the water to produce a concentrated acetate stream 138. The concentrated acetate stream 138 can be recycled to the acetate contactor 118 to increase the ethylene-acetate complex formation in the acetate contactor 118. The water vapor can be re-condensed separately to produce a water stream 140. The water stream 140 can be, for example, sent to a cooling tower to be used as utility in the plant or can be used as dilution steam.

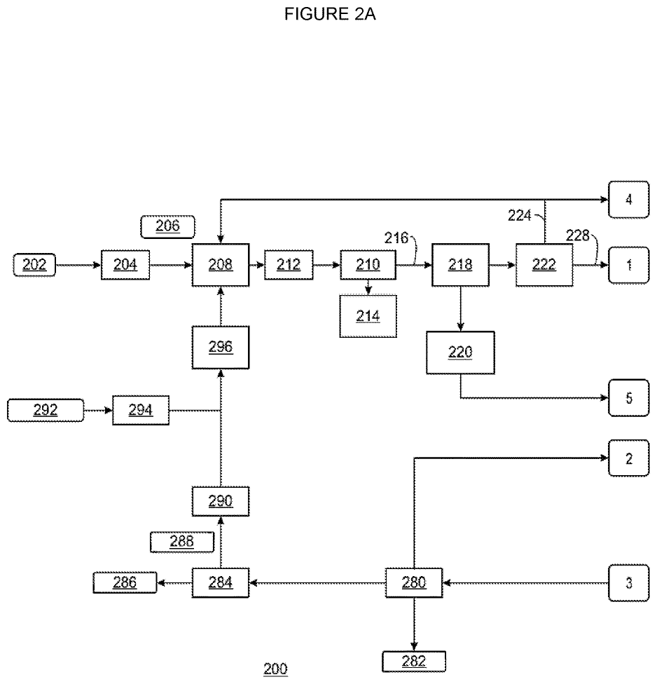

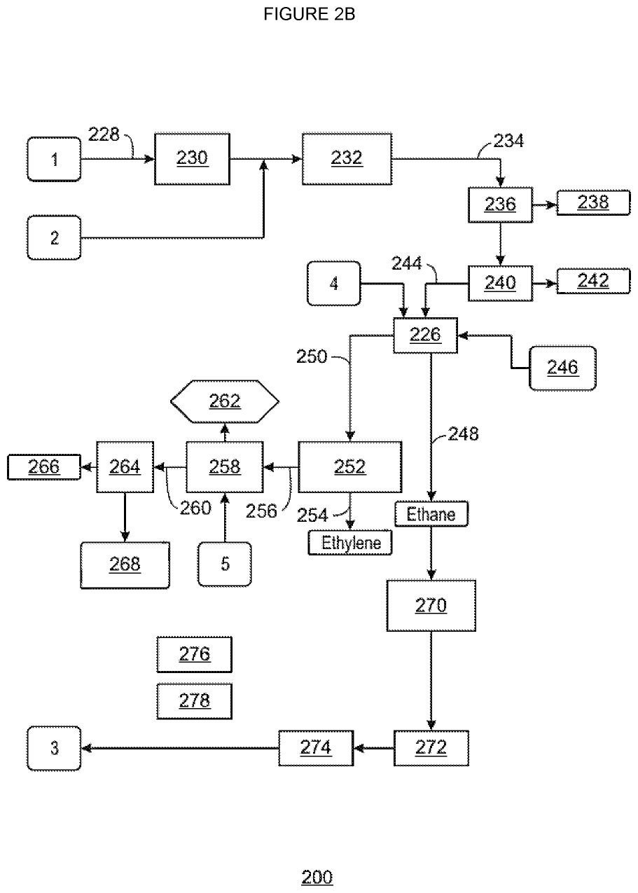

FIGS. 2A and 2B are block diagrams of an ODH system 200 for the ODH of ethane, in accordance with examples. The ethane ODH system 200 comprises an ODH reactor 210 comprising an ODH catalyst and configured to receive a feed stream comprising ethane 288 and an oxidant.

The oxidizing agent generally used in the process is air 202, although oxygen, generally mixed with a diluent, may also be used. The air 202 is flowed into an air separation unit (ASU) 204. In the ASU 204, the oxygen 206 is separated from other gases, such as nitrogen and carbon dioxide, among others. The oxygen 206 may then be mixed with a diluent, for example, in a steam dilution system 208.

To avoid process upsets, in many embodiments, mixtures of one or more alkanes with oxygen are employed using ratios that fall outside of the flammability envelope of the one or more alkanes and oxygen. In some embodiments, the ratio of alkanes to oxygen fall outside the upper flammability envelope. In these embodiments, the percentage of oxygen in the mixture can be less than 30 wt. %, in some cases less than 25 wt. %, or in other cases less than 20 wt. %, but greater than zero.

In embodiments with higher oxygen percentages, alkane percentages can be adjusted to keep the mixture outside of the flammability envelope. While a person skilled in the art would be able to determine an appropriate ratio level, in many cases the percentage of alkane is less than about 40 wt. % and greater than zero. As a non-limiting example, where the mixture of gases prior to ODH includes 20% oxygen and 40% alkane, the balance can be made up with an inert diluent. Non-limiting examples of useful inert diluents in this embodiment include, but are not limited to, one or more of steam, nitrogen, and carbon dioxide, among others. In some embodiments, the inert diluent exists in the gaseous state at the conditions within the reactor and does not increase the flammability of the ethane added to the reactor, characteristics that a skilled worker would understand when deciding on which inert diluent to employ. The inert diluent can be added to either of the alkane containing gas or the oxygen containing gas prior to entering the ODH reactor or may be added directly into the ODH reactor.

Although a number of different hydrocarbons may be used, in an ODH process, generally ethane is provided to the reactor along with oxygen. In some embodiments, the volumetric feed ratio of oxygen to ethane (O2/C2H6) provided to the one or more ODH reactors can be at least about 0.3, in some cases at least about 0.4, and in other cases at least about 0.5 and can be up to about 1, in some cases up to about 0.9, in other cases up to about 0.8, in some instances up to about 0.7 and in other instances up to about 0.6. The volumetric feed ratio of oxygen to ethane can be any of the values or range between any of the values recited above.

In some embodiments, mixtures that fall within the flammability envelope are employed, for example, in instances where the mixture exists in conditions that prevent propagation of an operational upset. In these non-limiting examples, the flammable mixture is created within a medium where ignition is immediately quenched. As a further non-limiting example, a user may design a reactor where oxygen and the one or more alkanes are mixed at a point where they are surrounded by a flame arresting material. Any ignition would be quenched by the surrounding material. Flame arresting materials include, but are not limited to, metallic or ceramic components, such as stainless steel walls or ceramic supports. In some embodiments, oxygen and alkanes are mixed at a low temperature, where an ignition event would not lead to an operational upset, then introduced into the reactor before increasing the temperature. The flammable conditions do not exist until the mixture is surrounded by the flame arrestor material inside of the reactor.

The amount of steam added to the process in the steam dilution system 208 affects the degree to which carbon dioxide acts as an oxidizing agent. In some embodiments, steam is added directly to the reactor 210, or steam is added to the individual reactant components—the lower alkane, oxygen, or inert diluent- or combinations thereof, and subsequently introduced into the reactor 210 along with one or more of the reactant components. Alternatively, steam may be added indirectly as water mixed with either the lower alkane, oxygen or inert diluent, or a combination thereof, with the resulting mixture being preheated before entering the reactor. When adding steam indirectly as water, a heater 212 is used to increase the temperature so that the water is converted to steam before entering the reactor.

Increasing the amount of steam added to the reactor 210 increases the degree to which carbon dioxide acts as an oxidizing agent. Decreasing the amount of steam added to the reactor 210 decreases the degree to which carbon dioxide acts as an oxidizing agent. The carbon dioxide present in the reactor 210 can be a product of an ODH reaction, added to the reactor 210 (for example, as part of the inert diluent), or a combination of both. In some embodiments a user monitors the carbon dioxide output and compares it to a predetermined target carbon dioxide output. If the carbon dioxide output is above the target a user can then increase the amount of steam added to the ODH process. If the carbon dioxide output is below the target a user can decrease the amount of steam added to the ODH process, provided steam has been added. Setting a target carbon dioxide output level is dependent on the requirements for the user. In some embodiments, increasing the steam added will have the added effect of increasing the amount of acetic acid and other by-products produced in the process. As larger amounts of acetic acid from the output of the ODH may be generated by higher levels of steam, reducing steam levels will decrease the amount generated. Conversely, higher levels of steam will increase the amount of carbon dioxide consumed.

In some embodiments, the amount of steam added to the reactor 210 is up to about 50 wt. %, in some circumstances up to about 40 wt. %, in some cases up to about 35 wt. %, in other cases up to about 30 wt. %, and in some instances up to about 25 wt. % and can be zero, in some cases at least 0.5 wt. %, in other cases at least 1 wt. %, in other cases at least 5 wt. %, in some instances at least 10 wt. % and in other instances at least 15 wt. % of the stream entering the reactor 210. The amount of steam in the stream entering the reactor 210 can be any value or range between any of the values recited above. As used herein, the reactor 210 includes a single reactor or multiple reactors.

In some embodiments, when using two or more reactors a user controls carbon dioxide output in only one, or less than the whole complement of reactors. For example, a user may opt to increase carbon dioxide output of an upstream reactor, relative to a downstream reactor, so that the higher level of carbon dioxide can be part of the inert diluent for the subsequent reactor. In that instance, maximizing carbon dioxide output upstream minimizes the amount of inert diluent that would need to be added to the stream prior to the next reactor.

There is no requirement for adding steam to the process, as it is one of many alternatives for the inert diluent. For processes where no steam is added, the carbon dioxide output is increased under the conditions used with respect to ethane, oxygen and inert diluent inputs. Decreasing the carbon dioxide output can then be a matter of adding steam to the reaction until carbon dioxide output drops to the desired level. In embodiments where oxidative dehydrogenation conditions do not include addition of steam, and the carbon dioxide output is higher than the desired carbon dioxide target level, steam may be introduced into the reactor while keeping relative amounts of the main reactants and inert diluent—lower alkane, oxygen and inert diluent—added to the reactor constant, and monitoring the carbon dioxide output, increasing the amount of steam until carbon dioxide decreases to the target level.

In some embodiments, a carbon dioxide neutral process is achieved by increasing steam added so that any carbon dioxide produced in the oxidative dehydrogenation process can then be used as an oxidizing agent such that there is no net production of carbon dioxide. Conversely, if a user desires net positive carbon dioxide output then the amount of steam added to the process can be reduced or eliminated to increase carbon dioxide production. As the carbon dioxide levels increase there is potential to reduce oxygen consumption, as carbon dioxide is competing as an oxidizing agent. The skilled person would understand that using steam to increase the degree to which carbon dioxide acts as an oxidizing agent can impact oxygen consumption. The implication is that a user can optimize reaction conditions with lower oxygen contributions, which may assist in keeping mixtures outside of flammability limits.

From the heater 212, the feed is introduced into the reactor 210. The reactor 210 may be any of the known reactor types applicable for a process, such as the ODH of alkanes. In some embodiments, the reactor 210 is a conventional fixed bed reactor. In a typical fixed bed reactor, reactants are introduced into the reactor at one end, and flow past an immobilized catalyst material, during which products are formed.

In some embodiments, the catalyst material includes molybdenum, vanadium, oxygen, and iron. The molar ratio of molybdenum to vanadium in the catalyst material can be from 1:0.1 to 1:0.5. The molar ratio of molybdenum to iron in the catalyst material can be from 1:0.25 to 1:5.5. Further, oxygen can be present in the catalyst material at least in amount to satisfy the valency of any present metal oxides.

In some embodiments, the catalyst material includes molybdenum, vanadium, oxygen, and aluminum. The molar ratio of molybdenum to vanadium can be from 1:0.1 to 1:0.5. The molar ratio of molybdenum to aluminum can be from 1:1.5 to 1:6.5. Further, oxygen can be present at least in an amount to satisfy the valency of any present metal oxides.

In some embodiments, the catalyst material includes molybdenum, vanadium, beryllium, and oxygen. The molar ratio of molybdenum to vanadium can be from 1:0.25 to 1:0.65. The molar ratio of molybdenum to beryllium can be from 1:0.25 to 1:85. Further, oxygen is present at least in an amount to satisfy the valency of any present metal oxides.

The products leave the reactor 210 at the opposite end from where the feed is introduced. Designing a fixed bed reactor suitable for the methods disclosed herein can follow techniques known for reactors of this type. A person skilled in the art would know which features are required with respect to shape and dimensions, inputs for reactants, outputs for products, temperature and pressure control, and means for immobilizing the catalyst material.

In some embodiments, the use of inert non-catalytic heat dissipative particles is used within one or more of the reactors. In various embodiments, the heat dissipative particles are present within the bed and include one or more non catalytic inert particulates having a melting point at least 30 °C, in some embodiments at least 250 °C, in further embodiments at least 500 °C above the temperature upper control limit for the reaction; a particle size in the range of 0.5 to 75 mm, in some embodiments 0.5 to 15 mm, in further embodiments in the range of 0.5 to 8 mm, in further embodiments in the range of 0.5 to 5 mm; and a thermal conductivity of greater than 30 W/mK (watts/meter Kelvin) within the reaction temperature control limits. In some embodiments the particulates are metal alloys and compounds having a thermal conductivity of greater than 50 W/mK (watts/meter Kelvin) within the reaction temperature control limits. Non-limiting examples of suitable metals that can be used in these embodiments include, but are not limited to, silver, copper, gold, aluminum, steel, stainless steel, molybdenum, and tungsten.

The heat dissipative particles can have a particle size of from about 1 mm to about 15 mm. In some embodiments, the particle size is from about 1 mm to about 8 mm. The heat dissipative particles can be added to the fixed bed in an amount from 5 to 95 wt. %, in some embodiments from 30 to 70 wt. %, in other embodiments from 45 to 60 wt. % based on the entire weight of the fixed bed. The particles are employed to potentially improve cooling homogeneity and reduction of hot spots in the fixed bed by transferring heat directly to the walls of the reactor. As described herein, in embodiments the reactor 210 may be cooled by the generation of high-pressure steam 214, for example, in a jacket around or coils within the reactor 210.

Additional embodiments include the use of a fluidized bed reactor, where the catalyst bed can be supported by a porous structure, or a distributor plate, located near a bottom end of the reactor and reactants flow through at a velocity sufficient to fluidize the bed (e.g. the catalyst rises and begins to swirl around in a fluidized manner). The reactants are converted to products upon contact with the fluidized catalyst and the reactants are subsequently removed from the upper end of the reactor. Design considerations those skilled in the art can modify and optimize include, but are not limited to, the shape of the reactor, the shape and size of the distributor plate, the input temperature, the output temperature, and reactor temperature and pressure control.

Some embodiments include using a combination of both fixed bed and fluidized bed reactors, each with the same or different ODH catalyst. The multiple reactors can be arrayed in series or in parallel configuration, the design of which falls within the knowledge of the worker skilled in the art.

In some embodiments, the stream exiting the one or more reactors is treated to remove or separate water and water-soluble hydrocarbons from the stream exiting the one or more reactors. In some embodiments, this stream is fed to a second reactor.

In some embodiments, the output stream exiting 216 the reactor 210 is directed to a quench tower 218 to be cooled and condensed. The quench tower 218 separates a stream of acetic acid and water 220 from the reactor output stream 216. The acetic acid stream 220 is fed to an extractant as shown in FIG. 2B.

The remaining gases from the quench tower 218 are fed to the acetic acid scrubber 222. The acetic acid scrubber 222 may remove traces of acetic acid, and other carbon compounds, from these gas streams by oxidation or adsorption.

The removed acetic acid and other carbon compounds exit the acetic acid scrubber 222 as a side stream 224. The side stream 224 is split, and one part of the side stream 224 is sent to an acetate absorber 226, and another part of the side stream 224 is sent to the steam dilution system 208.

A stream 228 containing unconverted lower alkane (such as ethane), corresponding alkene (such as ethylene), unreacted oxygen, carbon dioxide, carbon monoxide, optionally acetylene and inert diluent, are allowed to exit the acetic acid scrubber 222 and are fed to an oxygen removal system 230 (FIG. 2B).

The oxygenates removed via the quench tower 218 and/or acetic acid scrubber 222 can include carboxylic acids (for example acetic acid), aldehydes (for example acetaldehyde) and ketones (for example acetone). The amount of oxygenate compounds remaining in the stream 228 exiting the scrubber and fed to the oxygen removal system 230 will often be zero, for example, below the detection limit for analytical test methods typically used to detect such compounds. When oxygenates can be detected they can be present at a level of up to about 1 per million by volume (ppmv), in some cases up to about 5 ppmv, in other cases less than about 10 ppmv, in some instances up to about 50 ppmv and in other instances up to about 100 ppmv and can be present up to about 2 vol. %, in some cases up to about 1 vol. %, and in other cases up to about 1,000 ppmv. The amount of oxygenates or acetic acid in the stream exiting the scrubber and fed to the oxygen removal system 230 can be any value, or range between any of the values recited above.

In the oxygen removal system 230, as described herein, a high temperature membrane may be used to remove oxygen from the stream 228 exiting the acetic acid scrubber 222. The high temperature membrane may be heated by combusting access hydrocarbons in the stream 228, by combusting fuel added to the oxygen removal system 230, or both.

From the oxygen removal system 230, the stream 228 may be compressed, for example, in a first compressor system 232. The first compressor system 232 may include a single compressor or a series of compressors that sequentially boost the pressure of the stream 228.

The compressed stream 234 may then be fed to an amine scrubber 236 to remove CO2 238. From the amine scrubber 236, the compressed stream 234 may be fed to a caustic wash tower 240. The caustic wash tower 240 further reduces the concentration of CO2 in the compressed stream 234, sending the CO₂ in a rich caustic stream 242. The rich caustic stream 242 may then be treated to form a lean caustic stream which is returned to the caustic wash tower 240.

The caustic wash tower exit stream 244 is then fed to an acetate absorber 226. In this embodiment, the acetate absorber 226 is used to separate ethane from ethylene. Specifically, the acetate absorber 226 contains an acetate solution that absorbs the ethylene contained in the caustic wash tower exit stream 244. Most preferably, the acetate solution includes copper acetate. Silver acetate is one alternative acetate, although it is not as soluble in water as copper acetate. The acetate solution is added to the acetate absorber 226 by an acetate and water stream 246 as shown in FIG. 2B.

Ethylene absorption can be executed in the temperature range from −1 °C to 300 °C. The upper temperature limit is primarily defined by the thermal stability of the acetate. Copper acetate decomposes at temperatures higher than 168 °C to produce Cu2O and CuO. So the ethylene absorption step in the acetate absorber 226 is optimally operated at a temperature below 168 °C.

It is beneficial to operate the ethylene absorption step in the acetate absorber 226 at as high of a temperature as the thermal stability of the corresponding salt allows because a higher temperature results in a higher solubility. This allows for a higher ethylene capacity of the solution. A higher temperature also allows for faster absorption kinetics.

The downside of operating the ethylene absorption step at a higher temperature is greater thermal decomposition of the acetate salt and greater degradation of the absorbent. The degradation products cause undesired fouling of the absorption equipment.

The ethylene absorption step is performed at a temperature at which the ethylene-acetate complex remains stable and does not decompose/degrade. In some embodiments, the ethylene absorption step is operated at a temperature less than about 30 °C. In some embodiments, the ethylene absorption step is operated at a temperature greater than 10 °C. In some embodiments, the ethylene absorption step is operated at a temperature in a range of from 10 °C to about 30 °C. The ethylene absorption step is thus, in some embodiments, operated at 29 °C, to increase the benefits and decrease the downsides discussed above.

The degradation of the acetate solution can also occur as a result of a pH increase due to contaminants in the streams feeding the acetate absorber 226. For example, a trace amount of caustic from a caustic wash unit will react with copper acetate forming mixed copper oxides hydroxides and sodium acetate.

In the system disclosed herein, Cu20 or CuO that forms in the acetate absorber 226 is converted back to copper acetate by acetic acid. Ideally, all of the formed Cu20 or CuO is converted back. Acetic acid is fed to the acetate absorber 226 from the side stream 224 of the acetic acid scrubber 222. This is economically beneficial as acetic acid is an available by-product of the ODH reaction.

The acetate absorber 226 is optimally operated at a pressure up to 3000 kPa and most preferably 1564 kPa. The acetate absorber pressure is chosen such that the ethylene absorption rate, which increases with increased pressure, is high but the ethane dissolution rate, which also increases with increased pressure, is low.

Any type of acetate absorber 226 that achieves adequate mass transfer may be used. In some embodiments, the acetate absorber 226 is submerged in a water solution using a packed bed or bubble bed. This prevents dry metal acetylide from forming in the acetate absorber 226 and causing process upsets.

If the plant experiences significant carryover or accumulation of sodium in the acetate absorber 226, the acetate solution can be taken to a full or slip stream regeneration unit in which more caustic will be added to the solution. If the acetate used in the acetate absorber 226 is copper acetate, this allows CuO to precipitate. After, the CuO is filtered out of the solution. The CuO is next re-dissolved with an acetic acid solution to create copper acetate. The copper acetate can then be fed into the acetate absorber 226.

Once the ethylene is absorbed into the acetate, a stream containing mainly ethane 248 is also removed from the acetate absorber 226. A product stream 250 comprising absorbed ethylene, acetate, and water is also removed from the acetate absorber 226.

The product stream 250 is sent to a regenerator 252. The regenerator 252 removes the absorbed ethylene from the acetate. It also separates the ethylene from the acetate and water.

In some embodiments, the regenerator 252 is operated at 550 kPa and a reboiler duty of 9 GJ/hour. In some embodiments, the regenerator 252 is operated at a pressure in a range of from about 55 kPa to about 440 kPa. The regenerator 252 is further optimally operated up to 120 °C and most preferably at 90 °C. Lower pressures and higher temperatures may be used provided that the solution is not allowed to boil, which can cause fouling. Adjusting the pressure and temperature of the regenerator 252 can also affect the reboiler duty. Heat exchangers and pressure valves may be implemented in between the acetate absorber 226 and the regenerator 252 to achieve these operating parameters.

After separation in the regenerator 252, a stream of mainly ethylene 254 is removed and may be sold, depending on the on the required purity. A stream of concentrated acetate and water 256 is also removed from the regenerator 252.

The stream of concentrated acetate and water 256 is sent to an extractor 258.

The extractor 258 can be operated at a pressure in a range of from about 100 kPa to about 300 kPa. The extractor 258 can be operated at a temperature in a range of from about 20 °C to about 50 °C. In some embodiments, the extractor 258 is operated at 32 °C and 171 kPa. Heat exchangers and pressure valves may be implemented in between the regenerator 252 and the extractor 258 to achieve these operating parameters. Types of extractors include Cu-acetate-based, Na-acetate-based, or MTBE-based.

The extractor 258 outputs a diluted acetate stream 260 and an aqueous acetic acid stream 262. The aqueous acetic acid stream 262 is then sold or further purified. If the purity of the aqueous acetic acid stream 262 leaving the extractor 258 does not satisfy the end need, the aqueous acetic acid stream 262 is further purified by sending it to another acetic acid extractor.

The diluted acetate stream 260 is then sent to a dryer 264. The dryer 264 removes water 266 from the diluted acetate stream 260. The removed water 266 can be sent to a cooling tower to be used as utility within the plant or can be used as dilution steam. The dryer 264 can be operated at a temperature in a range of from about 100 °C to about 150 °C. The dryer 264 can be operated at a pressure in a range of from about 100 kPa to about 200 kPa. In some embodiments, the dryer 264 is operated preferably at 95 °C and 171.3 kPa.

A mixture 268 of acetate and water is also outputted from the dryer 264. The mixture 268 is ideally over 90 wt. % copper acetate and 10 wt. % water. This mixture 268 can be added to the acetate absorber 226 as a recycle stream.

If the ethane 248 obtained from the absorber 226 is to be further purified, it may be compressed in a second compressor system 270. The second compressor system 270 may include a single compressor or a chain of compressors that sequentially boost the pressure of the purified gas. The compressed purified gas may then be passed to a dryer 272 to remove excess water vapor. The dryer 272 may include molecular sieves to absorb the water, or may include a series of heat exchangers and chillers to physically condense the water, or both.

The dried stream is then passed to a chiller 274. The chiller 274 may include a series of heat exchangers, such as propane chilled heat exchangers, compressed nitrogen chilled heat exchangers, and heat exchangers cooled by fluids from other portions of the process. The chiller 274 may be integrated with, or feed, a depropanizer (C3R) 276, a deethanizer (C2R) 278, or both.

Returning to FIG. 2A, the chilled gas stream is fed to a demethanizer 280. From the demethanizer 280, an off gas stream 282 is sent to waste or to downstream processes. The off gas stream 282 includes the remainder of the inert diluent as well as methane removed from the chilled gas stream. Further, the demethanizer 280 returns a portion of the C2 compounds, such as ethylene and ethane, to the process upstream of the first compressor system 232. A C2 stream from the demethanizer 280 is fed to a C2 splitter 284.

The C2 splitter 284 divides the C2 stream into an ethylene product stream 286 and an ethane feed stream 288. The ethane feed stream 288 is vaporized in a heat exchanger 290 to form an ethane gas feed stream. An ethane feed 292 from another ethane source may be vaporized in a heat exchanger 294 and blended into the ethane gas feed stream.

The ethane gas feed stream is then passed through a high temperature heat exchanger 296 to be superheated. The superheated ethane gas feed stream is then fed to the steam dilution system 208 for use in the process.

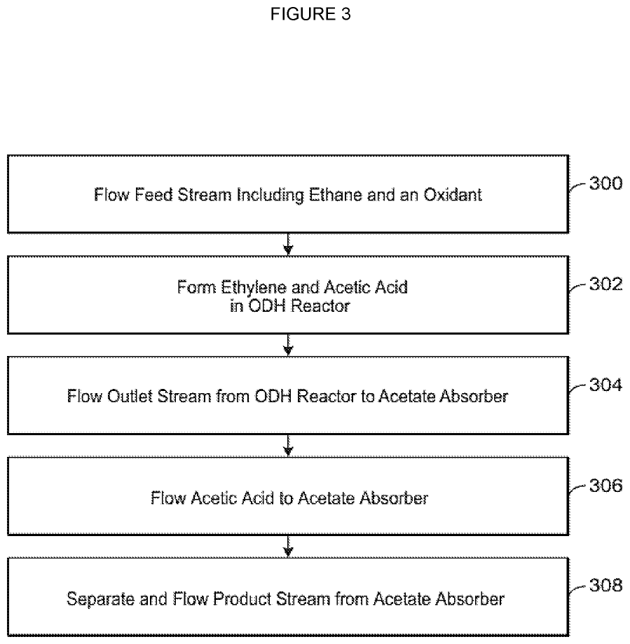

FIG. 3 is a block flow diagram of an example method 298 for implementing an acetate absorber into in an ODH system. The method 298 begins as block 300, when a feed stream including ethane and an oxidant is flowed to an ODH reactor. At block 302, ethylene and acetic acid are formed in the ODH reactor. The ODH reactor includes an ODH catalyst. The ethane and the oxidant react in the presence of the ODH catalyst under ODH conditions in the ODH reactor to form the ethylene and the acetic acid in the ODH reactor at block 302. At block 304, an outlet stream from the ODH reactor is flowed to an acetate absorber. The outlet stream flowed from the ODH reactor to the acetate absorber at block 304 includes the ethylene and the acetic acid. If a portion of the ethane from the feed stream does not react in the ODH reactor, the outlet stream includes residual ethane as well. In some embodiments, the outlet stream from the ODH reactor is cooled to a temperature less than an ethylene-acetate complexation temperature prior to being flowed to the acetate absorber at block 304. The acetate absorber includes an acetate that is capable of complexing double bonds (for example, a copper-based acetate or a silver-based acetate). The ethylene from the outlet stream complexes with the acetate in the acetate absorber to form an ethylene-acetate complex. At block 306, a stream including acetic acid is flowed to the acetate absorber. Finally, at block 308, a product stream is separated and flowed from the acetate absorber. Separating the product stream at block 308 can include directing the ethylene-acetate complex to an acetate regenerator and decoupling the ethylene and the acetate in the acetate regenerator to produce the product stream. The product stream flowed from the acetate absorber at block 308 includes the ethylene. In some embodiments, residual ethane from the outlet stream is recycled to the ODH reactor. For example, residual ethane from the outlet stream is combined with the feed stream to be recycled back to the ODH reactor.

### EXAMPLES

### Example 1 (Acetate Absorber Embodiment-Aspen Plus® Simulation)

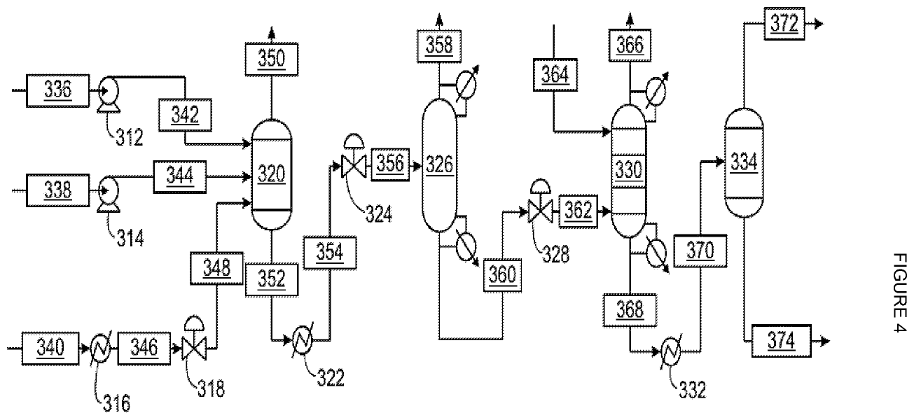

FIG. 4 is a simplified process flow diagram 310 of a process for implementing an acetate absorber in an ODH system. Aspen Plus® simulation (version 10.0, Aspen Technology, Inc.) was used to model the embodiment. The SR-POLAR equation of state was used for the simulation. The process includes a copper-acetate contactor 320, a copper-acetate regenerator 326, an acetic acid separator 330, and a copper-acetate knock off drum 334. A feed heat exchanger 316 heats the process gas 340 to produce a heated process gas 346. A control valve 318 controls flow of the heated process gas 348 to the bottom of the copper-acetate contactor 320. Fresh copper-acetate 338 flows to a pump 314. The pump 314 flows copper-acetate 344 to the top of the copper-acetate contactor 320 to remove ethylene from the heated process gas 348. The copper-acetate contactor 320 operates at 29 °C and 1,565 kPa. Ethane exiting the top of the copper-acetate contactor 320 is washed with water including a trace amount of acetic acid (for example, from the bottom of the acetic acid separator 330). In cases of operational upset in the copper-acetate contactor 320 leading to decomposition of the copper-acetate, a trace amount of acetic acid in the water can convert the decomposition product back to copper-acetate in the copper-acetate contactor 320. The gas 350 exiting the top of the copper-acetate contactor 320 includes about 92 wt. % ethane and the balance including methane, propane, and a trace amount of ethylene. In this example, an acetic acid solution 336 (including mostly water and some acetic acid) flows to a pump 312, and the pump 312 flows the acetic acid solution 342 to the copper-acetate contactor 320.

The bottom stream 352 exiting the bottom of the copper-acetate contactor includes about 20 wt. % ethylene, about 67 wt. % copper-acetate, and about 13 wt. % water and flows through a heat exchanger 322. The heat exchanger 322 heats the bottom stream 352 to produce a heated bottom stream 354. A control valve 324 controls flow of the heated bottom stream 356 to the copper-acetate regenerator 326, where ethylene is stripped from the copper-acetate. The copper-acetate regenerator 326 operates at 90 °C and 550 kPa. The reboiler duty of the copper-acetate regenerator 326 is about 9 gigajoules per hour (GJ/hr). The overhead product 358 exiting the top of the copper-acetate regenerator 326 is a high purity ethylene stream. The bottom stream 360 exiting the bottom of the copper-acetate regenerator 326 includes mostly copper-acetate and water.

A control valve 328 controls flow of the bottom stream 362 entering the acetic acid separator 330. The acetic acid separator 330 operates as an extraction column. An acetic acid stream 364 (including mostly water and some acetic acid) flows to the acetic acid separator 330 and contacts the bottom stream 362 inside the acetic acid separator 330 to separate acetic acid. The acetic acid separator 330 operates at 32 °C and 171 kPa. The overhead product 366 exiting the top of the acetic acid separator 330 is a high purity acetic acid stream. The bottom stream 368 exiting the bottom of the acetic acid separator 330 includes copper-acetate and water and flows through a heat exchanger 332. The heat exchanger 332 heats the bottom stream 368 to produce a heated bottom stream 370. The heated bottom stream 370 enters the copper-acetate knock off drum 334. The overhead stream 372 exiting the top of the copper-acetate knock off drum 334 is a high purity copper-acetate stream that includes some water. The overhead stream 372 can be condensed and recycled back to the copper-acetate contactor 320. The bottom stream 374 exiting the bottom of the copper-acetate knock off drum 334 is a high purity water stream. The bottom stream 374 can, for example, be sent to a cooling tower to be used as utility.

The energy/mass balances that resulted from this simulation are shown in Tables 1, 2, and 3 below. The simulation results of Example 1 show successful separation of ethylene by selectively dissolving ethylene in a copper-acetate solution. They also show that the removal of dissolved ethylene in copper-acetate in the regenerator is also possible by tuning operating conditions (e.g., pressure and temperature). Implementing the acetate absorber in the ODH system as shown in the process flow diagram 310 can, for example, be less energy intensive in comparison to traditional methods, such as cryogenic distillation.

TABLE 1

| | Units | 364 | 336 | 348 | 350 | 372 | 338 |
|---|---|---|---|---|---|---|---|
| From | | | | 318 | 320 | 334 | |
| To | | 330 | 312 | 320 | | | 314 |
| Temperature | °C | 35.0 | 35.0 | 130.3 | 57.0 | 95.0 | 44.0 |
| Pressure | kPa | 201.3 | 186.3 | 1600.0 | 1565.0 | 171.3 | 100.0 |
| Molar Vapor Fraction | | 0.0 | 0.0 | 1.0 | 1.0 | 1.0 | 0.0 |
| Mass Density | kg/cum | 974.7 | 983.2 | 13.9 | 17.8 | 5.7 | 1341.4 |
| Enthalpy Flow | GJ/hr | −624.7 | −25.9 | 22.6 | −35.5 | −61.6 | −74.6 |
| Average MW | | 20.4 | 18.3 | 28.3 | 28.8 | 98.5 | 99.5 |
| Mass Flows | kg/hr | 43078.0 | 1653.6 | 39838.3 | 12407.7 | 49615.6 | 49500.0 |
| CH4 | kg/hr | 0.0 | 0.0 | 677.1 | 677.1 | 0.0 | 0.0 |
| C2H4 | kg/hr | 0.3 | 0.0 | 27463.8 | 2.7 | 0.0 | 0.0 |
| C2H6 | kg/hr | 0.8 | 0.0 | 11633.3 | 11633.3 | 0.0 | 0.0 |
| C3H8 | kg/hr | 0.0 | 0.0 | 64.1 | 64.1 | 0.0 | 0.0 |
| CH3COOH | kg/hr | 7239.9 | 30.5 | 0.0 | 30.5 | 0.0 | 0.0 |
| H2O | kg/hr | 35837.0 | 1623.1 | 0.0 | 0.0 | 4615.6 | 4500.0 |
| CUACET | kg/hr | 0.0 | 0.0 | 0.0 | 0.0 | 45000.0 | 45000.0 |

TABLE 2

| | Units | 374 | 346 | 352 | 354 | 368 | 370 | 340 |
|---|---|---|---|---|---|---|---|---|
| From | | 334 | 316 | 320 | 322 | 330 | 332 | |
| To | | | 318 | 322 | 324 | 332 | 334 | 316 |
| Temperature | °C | 95.0 | 135.0 | 57.0 | 95.0 | 27.9 | 95.0 | 15.0 |
| Pressure | kPa | 171.3 | 2556.3 | 1565.0 | 1550.0 | 186.3 | 171.3 | 2571.3 |
| Molar Vapor Fraction | | 0.0 | 1.0 | 0.7 | 0.9 | 0.1 | 0.2 | 1.0 |
| Mass Density | kg/cum | 923.6 | 22.4 | 46.7 | 30.9 | 28.5 | 10.2 | 37.8 |
| Enthalpy Flow | GJ/hr | −581.7 | 22.6 | −43.8 | −32.0 | −665.5 | −643.7 | 12.9 |
| Average MW | | 18.0 | 28.3 | 50.2 | 50.2 | 33.7 | 33.7 | 28.3 |
| Mass Flows | kg/hr | 37344.5 | 39838.3 | 78584.2 | 78584.2 | 86960.0 | 86960.0 | 39838.3 |
| CH4 | kg/hr | 0.0 | 677.1 | 0.0 | 0.0 | 0.0 | 0.0 | 677.1 |
| C2H4 | kg/hr | 0.0 | 27463.8 | 27461.1 | 27461.1 | 0.0 | 0.0 | 27463.8 |
| C2H6 | kg/hr | 0.0 | 11633.3 | 0.0 | 0.0 | 0.0 | 0.0 | 11633.3 |
| C3H8 | kg/hr | 0.0 | 64.1 | 0.0 | 0.0 | 0.0 | 0.0 | 64.1 |
| CH3COOH | kg/hr | 0.0 | 0.0 | 0.0 | 0.0 | 0.0 | 0.0 | 0.0 |
| H2O | kg/hr | 37344.5 | 0.0 | 6123.1 | 6123.1 | 41960.1 | 41960.1 | 0.0 |
| CUACET | kg/hr | 0.0 | 0.0 | 45000.0 | 45000.0 | 45000.0 | 45000.0 | 0.0 |

TABLE 3

| | Units | 344 | 342 | 356 | 358 | 362 | 366 | 360 |
|---|---|---|---|---|---|---|---|---|
| From | | 314 | 312 | 324 | 326 | 328 | 330 | 326 |
| To | | 320 | 320 | 326 | | 330 | | 328 |
| Temperature | °C | 45.5 | 35.1 | 63.4 | 63.4 | 63.6 | 27.9 | 63.4 |
| Pressure | kPa | 1600.0 | 1600.0 | 550.0 | 550.0 | 201.3 | 186.3 | 550.0 |
| Molar Vapor Fraction | | 0.0 | 0.0 | 0.9 | 1.0 | 0.0 | 0.0 | 0.0 |
| Mass Density | kg/cum | 1338.3 | 983.1 | 11.0 | 5.6 | 1288.5 | 1068.6 | 1288.9 |
| Enthalpy Flow | GJ/hr | −74.6 | −25.9 | −32.0 | 52.9 | −98.4 | −57.3 | −98.4 |
| Average MW | | 99.5 | 18.3 | 50.2 | 28.1 | 87.0 | 60.0 | 87.0 |
| Mass Flows | kg/hr | 49500.0 | 1653.6 | 78584.2 | 27461.1 | 51123.1 | 7241.1 | 51123.1 |
| CH4 | kg/hr | 0.0 | 0.0 | 0.0 | 0.0 | 0.0 | 0.0 | 0.0 |
| C2H4 | kg/hr | 0.0 | 0.0 | 27461.1 | 27461.1 | 0.0 | 0.3 | 0.0 |
| C2H6 | kg/hr | 0.0 | 0.0 | 0.0 | 0.0 | 0.0 | 0.8 | 0.0 |
| C3H8 | kg/hr | 0.0 | 0.0 | 0.0 | 0.0 | 0.0 | 0.0 | 0.0 |
| CH3COOH | kg/hr | 0.0 | 30.5 | 0.0 | 0.0 | 0.0 | 7239.9 | 0.0 |
| H2O | kg/hr | 4500.0 | 1623.1 | 6123.1 | 0.0 | 6123.1 | 0.0 | 6123.1 |
| CUACET | kg/hr | 45000.0 | 0.0 | 45000.0 | 0.0 | 45000.0 | 0.0 | 45000.0 |

What is claimed is:

1. A process for the oxidative dehydrogenation of ethane, the process comprising:
contacting an ethane feed with an oxidant in the presence of an oxidative dehydrogenation catalyst in an oxidative dehydrogenation reactor under oxidative dehydrogenation conditions to produce an output stream, wherein the ethane feed comprises ethane, and the output stream comprises ethylene, acetic acid, and residual ethane from the ethane feed; and
contacting the output stream with an acetate in an absorber to separate a product stream from a residual ethane stream, wherein the product stream comprises the ethylene, and the residual ethane stream comprises the residual ethane from the ethane feed.

2. The process of claim 1, comprising, prior to contacting the output stream with the acetate in the absorber, cooling the output stream to a temperature at which the ethylene forms a complex with the acetate in response to contacting the acetate.

3. The process of claim 2, wherein an ethylene-acetate complex is formed in the absorber in response to the output stream contacting the acetate in the absorber, and the process comprises decoupling the ethylene-acetate complex in an acetate regenerator to form the product stream.

4. The process of claim 3, comprising:
prior to contacting the output stream with the acetate in the absorber, directing the output stream to an acetic acid scrubber upstream of the absorber;
separating a side stream comprising at least a first portion of the acetic acid from the output stream using the acetic acid scrubber; and
directing the side stream to the absorber.

5. The process of claim 4, wherein decoupling the ethylene-acetate complex in the acetate regenerator forms an acetate stream comprising water and the acetate.

6. The process of claim 5, comprising directing the acetate stream to an extractor to separate an aqueous acetic acid stream and a diluted acetate stream.

7. The process of claim 6, comprising:
directing the diluted acetate stream to a dryer to separate a recycle stream comprising the acetate; and
directing the recycle stream to the absorber.

8. The process of claim 6, comprising:
prior to directing the output stream to the acetic acid scrubber, directing the output stream to a quench tower upstream of the acetic acid scrubber;
separating a bottom stream comprising at least a second portion of the acetic acid from the output stream using the quench tower; and
directing the bottom stream to the extractor.

9. The process of claim 1, wherein the oxidative dehydrogenation catalyst comprises molybdenum, vanadium, oxygen, and an element chosen from at least one of iron, aluminum, or beryllium.

10. The process of claim 1, wherein the acetate is copper acetate.

11. The process of claim 1, wherein the acetate is silver acetate.

12. A system for the oxidative dehydrogenation of ethane, comprising:
a feed stream comprising an oxidant and ethane;
an oxidative dehydrogenation reactor comprising an oxidative dehydrogenation catalyst, wherein the oxidative dehydrogenation reactor is configured to receive the feed stream and contact the feed stream with the oxidative dehydrogenation catalyst under oxidative dehydrogenation conditions to produce an output stream comprising ethylene, acetic acid, and residual ethane from the feed stream;
an absorber comprising acetate, wherein the absorber is configured to receive the output stream and separate a product stream from the output stream, the acetate is configured to complex with the ethylene to form an ethylene-acetate complex, and the product stream comprises the ethylene.

13. The system of claim 12, comprising a quench tower downstream of the oxidative dehydrogenation reactor and upstream of the absorber, the quench tower configured to receive the output stream and cool the output stream to a temperature at which the ethylene forms the ethylene-acetate complex with the acetate in response to contacting the acetate in the absorber.

14. The system of claim 13, comprising an acetic acid scrubber upstream of the absorber and downstream of the quench tower, wherein the acetic acid scrubber is configured to receive the output stream from the quench tower and separate the output stream into a side stream and an acetic acid scrubber exit stream, the side stream comprises at least a first portion of the acetic acid from the output stream, the acetic acid scrubber exit stream comprises the ethylene and the residual ethane from the feed stream, and the absorber is configured to receive the side stream and the acetic acid scrubber exit stream.

15. The system of claim 14, wherein the absorber comprises a regenerator configured to decouple the ethylene-acetate complex to form the product stream and an acetate stream comprising water and the acetate.

16. The system of claim 15, comprising an extractor configured to receive the acetate stream and separate the acetate stream into an aqueous acetic acid stream and a diluted acetate stream.

17. The system of claim 16, comprising a dryer configured to receive the diluted acetate stream to separate the diluted acetate stream into a recycle stream comprising the acetate and a dryer exit stream comprising water, wherein the absorber is configured to receive the recycle stream from the dryer.

18. The system of claim 17, wherein the quench tower is configured to contact the output stream with a quench stream to cool the output stream, the quench tower is configured to discharge a bottom stream comprising at least a portion of the acetic acid from the output stream, and the extractor is configured to receive the bottom stream from the quench tower.

19. The system of claim 12, wherein the oxidative dehydrogenation catalyst comprises molybdenum, vanadium, oxygen, and an element chosen from at least one of iron, aluminum, and beryllium.

20. The system of claim 19, wherein the acetate of the absorber is copper acetate or silver acetate.
